(19) United States
(12) Patent Application Publication
(10) Pub. No.: US 2008/0119735 A1
(43) Pub. Date: May 22, 2008

(54) Ultrasound imaging system and method with offset alternate-mode line

(75) Inventors: Shengtz Lin, Cupertino, CA (US); Hue Phung, Cupertino, CA (US)

Correspondence Address: Bo-In Lin, 13445 Mandoli Drive, Los Altos Hills, CA, 94022, US

(73) Assignee: SonoWise, Inc.

(21) Appl. No.: 11/601,991

(22) Filed: Nov. 20, 2006

(51) Int. Cl. A61B 8/12

(52) U.S. Cl. 600/450; 600/437; 600/443

(57) ABSTRACT

A method for generating a dedicated M beam profile which allows the user to offset the beam origin or steering angle for the cardiology application; display the M line with or without the B mode on the screen; and the M line does not use any of the same beam profile from B line, or is created out of the acquired B lines as a virtual M line.

### BACKGROUND

The present invention relates to ultrasound imaging. Embodiments of the present invention are especially suitable for ultrasound imaging that include Motion-mode (M-mode) imaging.

Ultrasound imaging systems are known. For example, medical ultrasound imaging is discussed in U.S. Pat. No. 5,345,939, RE37,088E (Reissue of U.S. Pat. No. 5,515,856), U.S. Pat. No. 6,248,071, and U.S. Pat. No. 6,783,497. Conventional details of ultrasound imaging systems need not be described in the present document.

Ultrasound imaging systems use a variety of imaging modes. For example, Brightness-mode (B-mode) imaging and M-mode imaging are frequently used in, for example, medical ultrasound imaging for cardiology.

FIG. 1A schematically illustrates B-mode imaging. In B-mode imaging, a probe 118a, which is part of an ultrasound imaging system, emits beams of ultrasonic energy, one beam at a time, each along one of multiple adjacent scan lines 110. For the purpose of clarity of illustration, FIG. 1A only shows a few exemplary scan lines 110. The scan lines 110 define a scan plane 114 as a cross sectional plane cut through a subject, e.g., a patient's heart 116 as shown in two-dimensional cross section, under a medial examination. The ultrasound imaging system receives echoes from the beams of ultrasonic energy and processes the echoes to reconstruct an image that depicts at least a portion of the scan plane 114, including a cross-section image of at least part of the subject, e.g., the patient's heart 116. Typically, the two dimensional image is successively updated in time to obtain video display. A common type of B-mode imaging uses a sector scanning approach, shown in FIGS. 1A and 1B, in which the multiple adjacent scan lines 110 are at different angles and sweep out a substantially “pie-slice” shaped scan plane 114.

A typical medical ultrasound imaging system employs an array of individual transducer elements in a “probe” to generate the individual ultrasonic beams. The array of transducer elements may be a flat phase array 118a (FIG. 1A) or a curved linear array 118b (FIG. 1B). The flat array 118a of FIG. 1A is used to generate a substantially pie-shaped, sector-scanning scan plane uses phased-array methodology to generate the individual ultrasonic beams that each have the angle of one of the scan lines 110. In other words, the flat array 118a uses phased-array methodology to steer each individual ultrasonic beam (and also to focus each individual ultrasonic beam).

M-mode imaging is often used in conjunction with B-mode imaging, especially in the field of cardiology. In conventional M-mode imaging, successive ultrasonic pulses are sent along a single scan line. Each pulse (or an average of several successive pulses) produces echoes that are analyzed to produce a linear sliver of image, and these slivers of image are cumulatively displayed along a time axis in a display (e.g., a printout and/or a video display). An M-mode image shows the movement over time, along the scan line, of features being examined. M-mode imaging is used, for example, in cardiology to observe the opening and closing of heart valves over time. FIG. 2 schematically illustrates an example M-mode image 210 in which the horizontal axis is the time axis.

In conventional M-mode imaging, the single scan line used for the M-mode imaging has substantially the same position as one of the scan lines that define the scan plane used for B-mode sector scanning. For example, the M-mode ultrasound pulses and the B-mode ultrasound pulses are positioned as if they originate from a same apex point (or, origin) of the B-mode “pie slice”. For example, in FIG. 1A (or 1B), an M-mode scan line would look like, e.g., the line 112a (or 112b). As can be seen from, e.g., FIG. 1B, the apex point of a B-mode sector image might be a virtual apex located by hypothetically extending scan lines until they meet.

In conventional M-mode imaging, a method, as now discussed in connection with FIG. 3, has been proposed to obtain a “virtual M-mode line” 310 that does not necessarily have substantially the same original position as any of the pulse lines that define the B-mode scan plane. That method is described in U.S. Pat. No. RE37,088E. Essentially, the method requires very high-frame-rate 2D image acquisition and storage of a very large amount of 2D B-mode image data in a buffer, in order that data along the virtual M-line can be selectively extracted from the 2D data set. While this method provides improved flexibility in choice of (virtual) M-mode line position, it requires a sophisticated system architecture and a powerful processing engine and a very high frame rate of 2D image acquisition, for example, at least a hundred 2D frames per second for an M-mode vector update rate of once every 10 milliseconds (ms).

### SUMMARY OF THE INVENTION

What is needed are apparatuses and methods to provide flexibility of M-mode line selection in ultrasound imaging while avoiding at least some of the drawbacks of the “virtual M-mode line” methodology. For example, it is desirable to avoid high system complexity or high system cost or very high ultrasound frame rate.

According to some embodiments of the present invention, the invention discloses a method for generating a dedicated M beam profile which allows the user to offset the beam origin or steering angle for the cardiology application, and display the M line with or without the B mode on the screen; and the M line does not use any of the beam profile from B line or is created out of the acquired B lines as the virtual M line.

### BRIEF DESCRIPTION OF THE DRAWINGS

In order to more extensively describe some embodiment(s) of the present invention, reference is made to the accompanying drawings. These drawings are not to be considered limitations in the scope of the invention, but are merely illustrative.

FIG. 1A is a schematic diagram of a flat array of ultrasound transducer elements; the flat array sweeping out a sector-scanning B-mode scan plane, the scan plane including an M-mode scan line having substantially the same position as a scan line of the B-mode scan plane, according to the conventional art.

FIG. 1B is like FIG. 1A, but with a curved array of ultrasound transducer elements.

FIG. 2 is a schematic diagram of an example M-mode image.

FIG. 3 is a schematic diagram of a flat array of ultrasound transducers; the flat array sweeping out a sector-scanning B-mode scan plane, the plane including a “virtual M-mode line”, according to a complicated and resource-intensive methodology described in the Background section above.

FIG. 4 is a schematic diagram of an ultrasound imaging system according to an embodiment of the present invention, the system being configured to implement methodology of an embodiment of the present invention.

FIG. 5A is a schematic diagram showing beam forming for ultrasound transmission using phased-array methodology.

FIG. 5B is a schematic diagram showing beam forming for ultrasound echo reception using phased-array methodology.

FIG. 6A is a schematic diagram of a flat array of ultrasound transducer elements; the transducers sweeping out a sector-scanning B-mode scan plane, the plane including an offset M-mode scan line, according to an embodiment of the present invention.

FIG. 6B is like FIG. 6A, but with a curved array of ultrasound transducer elements.

FIG. 7A is a schematic diagram showing the geometry of a B-mode scan plane and an offset M-mode scan line, for a flat array of ultrasound transducer elements, according to an embodiment of the present invention.

FIG. 7B is like FIG. 7A, but with a curved array of ultrasound transducer elements.

FIG. 8 schematically illustrates an example Doppler-mode image in which the horizontal axis is the time axis.

FIG. 9 is a schematic diagram of a flat array of ultrasound transducer elements; the transducers sweeping out a sector-scanning B-mode scan plane, the plane including an offset Doppler-mode scan line, according to an embodiment of the present invention.

FIG. 10 is a schematic flow diagram that illustrates one method according to an embodiment of the present invention for conducting ultrasound imaging in a first ultrasound-imaging mode that highlights an elongated region within a larger region, the larger region being imaged according to a second ultrasound mode.

### DETAILED DESCRIPTION OF SPECIFIC EMBODIMENTS

The description and the drawings of the present document describe examples of some embodiments of the present invention and also describe some exemplary optional features and/or alternative embodiments. It will be understood that the embodiments described are for the purpose of illustration and are not intended to limit the invention specifically to those embodiments. Rather, the invention is intended to cover all that is included within the spirit and scope of the invention, including alternatives, variations, modifications, equivalents, and the like. Use of language in the present document is not intended for misinterpreting as to limit the scope of the invention.

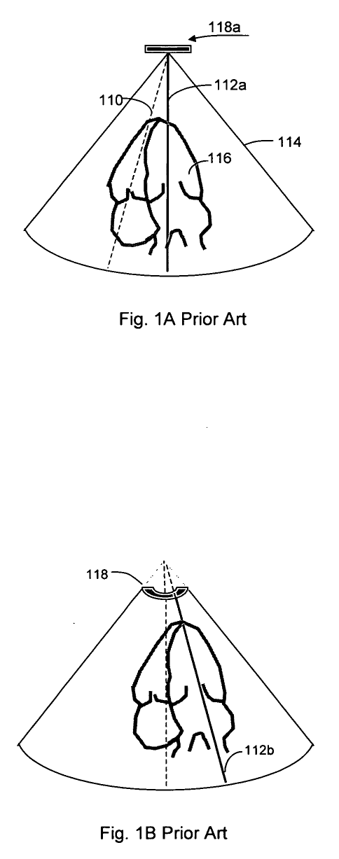

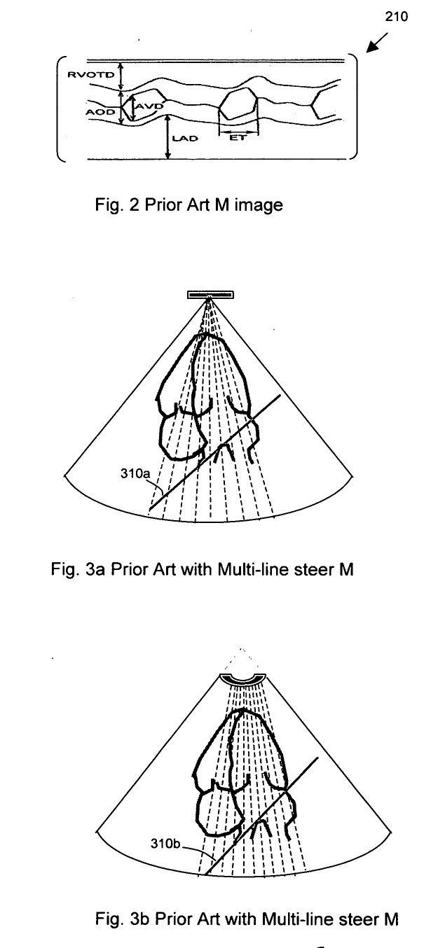

Preliminarily, it may be helpful to very briefly further summarize some basics of ultrasound imaging. Referring again to FIG. 1A, recall that, in B-mode imaging, a probe 118a emits beams of ultrasonic energy, one beam at a time, each along one of multiple adjacent scan lines 110. As each ultrasonic beam travels along its path, it is successively partial reflected, or echoed, by the material through which it travels. Different materials, or interfaces between different materials, generate echoes having different intensities. From a single beam, the echoes from successively farther away (from the probe 118a) are successively received by the probe 118a. These echoes are collected into a vector. Earlier received echoes correspond to physical features nearer to the probe 118a, and later received echoes correspond to physical features farther from the probe 118a. The ultrasound imaging system processes the vector of received echo information from each beam to produce a 2D image, corresponding to a single scan plan, for display. In color flow imaging system, the ultrasound imaging system actually sends multiple ultrasonic beams in quick succession over the single scan line 110 and processes the separately collected vectors of echoes from the multiple beams with autocorrelation algorithm, and uses the resulting processed vector to produce the color flow image. In both B or color flow mode, the multiple slivers of image corresponding to multiple scan lines are visually combined on a display to form a two dimensional image. Typically, the two dimensional image is successively updated in time to obtain video display.

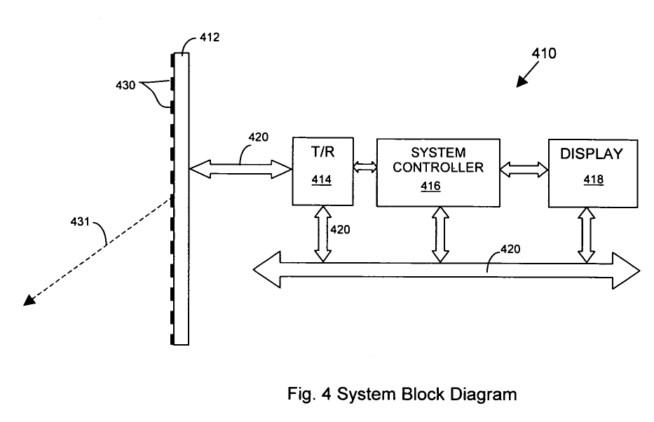

FIG. 4 is a schematic diagram of an ultrasound imaging system 410 according to an embodiment of the present invention. The system 410 is configured to implement methodology of an embodiment of the present invention. The ultrasound imaging system 410 includes a transducer subsystem 412, a transmitter/receiver (T/R) subsystem 414, a system controller 416, a display 418, and various data and control paths 420 that interconnect the various portions of the ultrasound imaging system 410. The componentry and imaging algorithms of the system 410 can be of any effective conventional type, as long as they are further configured to implement methodology according to an embodiment of the present invention.

In the example configuration shown in FIG. 4, the transducer subsystem 412 includes multiple individual transducers elements 430 (for example, about 64 or about 128 or about some other number of transducers) arranged in an array. The array is shown in FIG. 4 as a flat array, but it may instead be another arrangement, e.g., curved. The T/R subsystem 414 is coupled to the transducer subsystem 412 and is configured to operate the transducer subsystem 412 to generate beams of ultrasonic acoustic energy, generally one at a time. The T/R subsystem 414 is configured to then receive echoes of the beam right after transmitting, also via the transducer subsystem 412, in order for the system controller 416 to process the echo information to form image information for display by the display 418. The system controller 416 includes data storage, at least one data processor for processing the information data received from a scanning operation, and software stored on the data storage configured to instruct the information processor to execute includes methodology according to an embodiment of the present invention. The software is also an embodiment of the present invention.

Preferably, the system 410 is configured to perform scanning that substantially uses adjacent nonparallel scan lines to form multidimensional scan images. For example, the system 410 is preferably configured to perform scanning that is substantially two-dimensional and substantially sector. For performing B-mode sector scanning, a flat transducer subsystem 412 as shown in FIG. 4 would employ conventional phased-array methodology in order to steer a beam of acoustic energy in a desired direction 431. The schematic icons 432 of pulses shown in FIG. 4 schematically (but not precisely) convey the essence of phased-array beam forming: namely, that a pulsed waveform for forming an ultrasound beam is delayed (or phase shifted) by different amounts to different individual transducer elements 430, in order to steer (and focus) the beam. The schematic icons 432 similarly convey that, in typical phased-array methodology, received echoes are also combined using variable delays, in order to focus the formed image along the direction 431 of the beam of acoustic energy. Some basic aspects of phased-array methodology will be briefly further mentioned below.

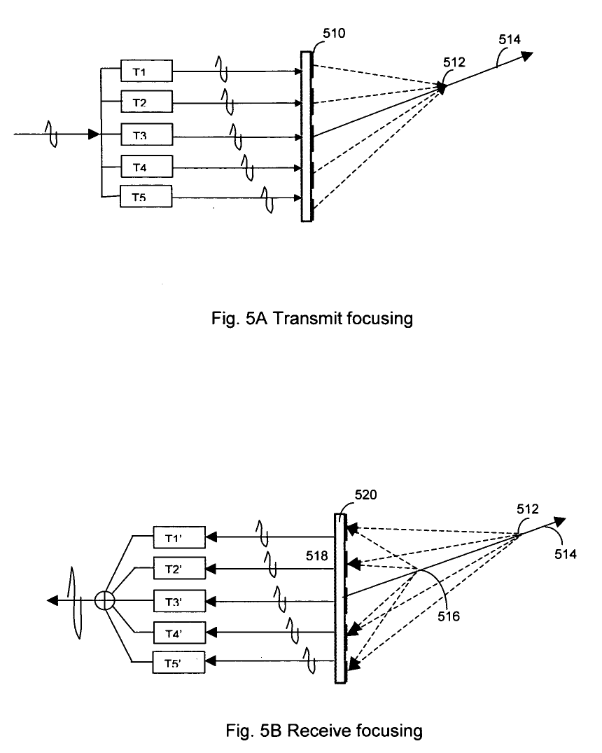

Although phased-array methodology, in general, is a conventional art, it is helpful to briefly discuss some of its ideas herein. FIG. 5A is a schematic diagram showing phased-array beam forming for B-mode ultrasound transmission. In FIG. 5A, only 5 transducer elements 510 are shown and discussed, for simplicity, with the understanding that the actual number of transducer elements is typically much higher, e.g., 64 or 128 or some other number. In beam forming for transmission, a pulse waveform is sent, with mathematically chosen delays T1, T2, . . . T5, to transducer elements 510. Each transducer element 510 in response emits an ultrasonic acoustic pulse, delayed by its particular delay T1, . . . or T5. The ultrasonic pulses, together, are said to form a beam of ultrasonic acoustic energy. The ultrasonic pulses are timed to arrive approximately simultaneously at a desired focus point 512 along a desired beam path 514. The timing is possible given a known speed of sound through the anticipated material being probed, and given the focus point 512 and beam path 514. For medical ultrasound imaging, it is considered sufficiently (though not literally) accurate and precise to say that the speed of sound is 1540 meters/second for soft tissue through a patient being probed, and to say that the speed of sound is constant throughout the patient. For improved control of side-lobe levels and other acoustic signal characteristics, in addition to variable delays, the amplitudes of the acoustic transmissions from individual transducer elements 510 can also be variably controlled (apodized), in a conventional fashion. Various apodization schemes exist; for example, the Hamming window is a common scheme. Frequently, for simplicity, apodization is not used for pulsed-waveform transmission, but is used in echo receiving, further described below.

FIG. 5B is a schematic diagram showing reception of incoming echoes created by an emitted ultrasonic beam. The emitted beam was directed along the beam path 514. The emitted beam was formed, for example, according to the discussion of FIG. 5A. The reception of echoes from the emitted beam is intended to capture information about physical features along the beam path 514. The goal is for the echo created at substantially each particular point on the beam path 514, such as point 516, to be received by multiple transducer elements, and for those received signals to be combined to obtain constructive acoustic interference.

Whatever the angle of the beam path 514, the echo from any particular point 516 on the beam path 514 is not expected to arrive at all transducer elements 510 simultaneously because the travel distance from the particular point 516 to each transducer element will generally differ. The ultrasonic imaging system has data to determine the distances between any given points and any given probe is provided with data to determine the approximate/assumed speed of sound through the medium being probed, and also has data of the timing of the emitted beam. The ultrasonic imaging system therefore has data to determine the complete schedule of when an echo from each point along the beam path 514 should arrive at each transducer element. Therefore, the ultrasonic imaging system can be, and is, programmed to combine the echo signal values received at the various transducer elements with selective delays, such that image information corresponding to various points along the beam path 514 can be obtained. The selective delays vary with time, according to the geometry corresponding to the particular point whose echoes are being combined.

In addition to selective delays, selective gains (e.g., a window function, e.g., a Hamming window) can be applied (apodization) to the different transducer elements, in conventional fashion. For example, typically, for receiving echoes from points very near the transducer-element array, far-away transducer elements may be not used. The selective gains typically vary with time, according to the geometry corresponding to the particular point whose echoes are being combined. Normally, the f-number is used to define the ratio of the focal length and aperture size.

The above brief discussion of some ideas from phased-array methodology is for convenience only, and is not intended to be complete or limiting. Again, any effective conventional ultrasound technology can be used in the present invention, so long as the conventional technology is further configured to include methodology, further discussed below, according to the present invention.

According to some embodiments of the present invention, an ultrasound imaging system and method are capable of producing M-mode images even for an M-mode line that the origin is offset from the B-mode scan lines that form the B-mode scan plane. For example, an M-mode line may be realized (1) that does not share a common origin point, actual or virtual, with the B-mode sector-scanning scan lines that form the B-mode scan plane, or (2) that does not have about the same position as any of the B-mode sector-scanning scan lines. Any effective method can be used to obtain imaging along the offset M-mode line, other than exactly the “virtual M-mode line” technology discussed in the above Background section. In a preferred embodiment, the ultrasound image system and method are configured for use in medical cardiology imaging diagnosis.

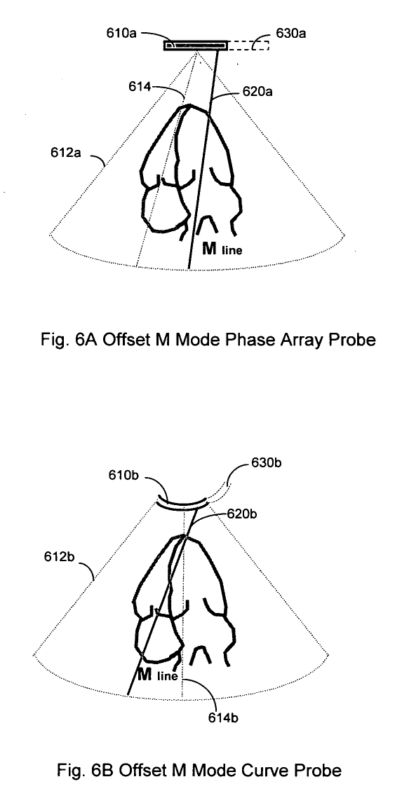

FIG. 6A is a schematic diagram of a flat phase array 610 of ultrasound transducer elements operating according to such some embodiment of the present invention. The desired M-mode line is preferably indicated and chosen by the ultrasound operator via a user interface, relative to a B-mode image being presented to the ultrasound operator in the user interface. The user interface is provided on a display screen, under control of the processor of the ultrasound imaging system. The ultrasound imaging system may, for example, include the elements shown in FIG. 4.

In FIG. 6A, the ultrasound imaging system operates to define a B-mode scan plane 612a. For example, the ultrasound imaging system may operate its array 610a of transducer elements to perform conventional phased-array B-mode sector scanning. The ultrasound imaging system further operates its array 610a of transducer elements to obtain M-mode imaging along an offset M-mode line 620a.

As is schematically shown in FIG. 6A, the offset M-mode line 620a does not share a common origin point with the B-mode scan lines, such as a scan line 614a, that define the B-mode scan plane. The M-mode line 620a also does not have a same focus beam profile as any of the B-mode scan lines. For example, in one embodiment, the individual B-mode scan lines, such as scan line 614a, are positioned to have an origin (e.g., even an imagined best approximate point of convergence) nearest to the physical center of the array 610a, and the offset M-mode line 620a is capable of being selected and realized, through different beam profile, to have an origin that is not nearest to the physical center of the phased array 610.

Any effective method can be used to obtain imaging along the offset M-mode line 620a, other than exactly the “virtual M-mode line” technology discussed in the above Background section. For example, (1) the M-mode imaging is preferably conducted to include using an dedicated beam of ultrasonic energy transmitted substantially along the offset M-mode line 620a ; or (2) the offset M-mode scanning is preferably conducted to include using a set of delay profile (and, optionally, gain) parameters that is different from such parameters of the B-mode beam along the nearest B-mode scan line; or (3) the M-mode imaging is preferably conducted without requiring a 2D B-mode imaging frame rate of at least about the M-mode refresh rate, or even without requiring a 2D B-mode imaging frame rate of at least about half the M-mode refresh rate. Typical B mode frame rate in cardiac application is from 20-60 frames per second and preferably 40 frames or higher. A preferred M mode refresh rate is about 100-400 vector per second, typically 200, or 5 ms interval per M refresh. For an operation to extract M out of B mode, then B frame rate needs to be significantly increased, which will complicate the system design; or (4) the M-mode imaging is preferably conducted to include obtaining each sliver of M-mode image along the time axis substantially without combining image data from ultrasonic beams separately sent along multiple scan lines that intersect the M-mode line at different points.

For example, variable delays (and, optionally, gains) for individual transducer elements may be computed for the M-mode beam, in standard beam-forming fashion, using an offset point as the origin or center of the desired M-mode beam. Because the origin of the desired M-mode beam need not be the center of the phased array 610a of transducer elements, the standard computed variable delays (and, optionally, gains) algorithm, may include delays for nonexistent transducer elements 630a beyond the actual edge of the phased array 610a. Such variable delays (gains) for the non-existing elements may simply be ignored, leaving an unsymmetrical number of transducer elements to each side of the origin of the M-mode beam. Such asymmetry is more than likely to lead to additional asymmetry in the shape of the formed M-mode beam and the sizes of its side lobes, but the formed beam at the offset origin and steering angle can still be used. According to design choice, the M-mode beam may be constrained, e.g., constrained during M-mode scan line setting by a human operator, to have an origin that permits at least a minimum number or fraction, e.g., a predetermined number or fraction, of transducers to actually exist on the shorter side of the origin. For example, the minimum number may be required to be nonnegative, which is a requirement that the M-mode scan line must intersect with the array 610a of transducer elements. The capability of this beam origin offset with angle steering provides benefit to the patient with slightly deviated heart orientation.

FIG. 6B is like FIG. 6A, but with a curved array 610b of ultrasound transducer elements. Beam forming for curved transducer arrays is known, and the above discussion relative to FIG. 6A applies also to FIG. 6B, with the understanding that FIG. 6B shows a curved, not flat array of ultrasound transducer elements. FIG. 6B shows a sector-scanning, B-mode scan plane 612b, defined for example by the different angles of successive individual B-mode scan lines 614b. In a simple embodiment, the B-mode scanning does not use phased-array beam forming for steering the angle of the B-mode beams. Instead, the B-mode scanning simply uses curved-linear-array methodology using a curved transducer array 610b whose curvature matches the desired curvature of the sector-scanning scan plane 612b and the same beam profile is used across every B line. However, the M-mode scanning does use phased-array beam forming algorithm to position and steer the M-mode scanning beam along the user-chosen M-mode line 620b. Nonexistent transducer elements 630b beyond the actual edge of the curved phased array 610b are shown for the same purpose of discussion, as were similarly nonexistent transducer elements 630a in FIG. 6A.

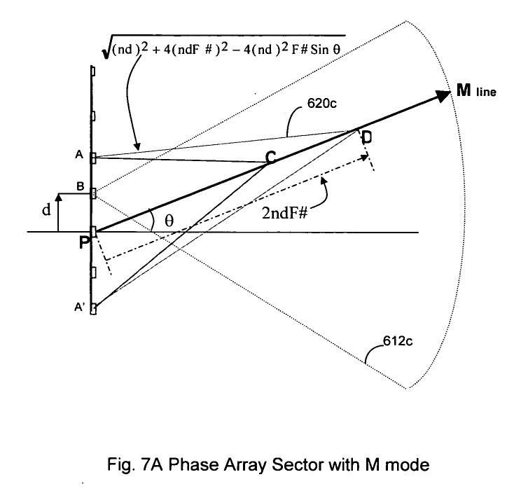

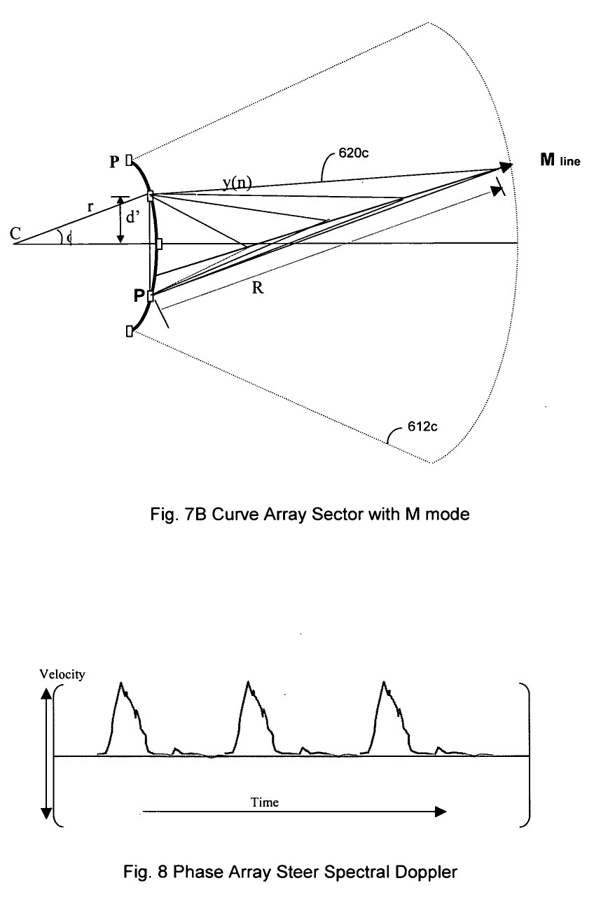

FIG. 7A is a schematic diagram for showing the geometry of a B-mode scan-plane 612c and an offset M-mode line 620c, according to an embodiment of the present invention. Example points A, A′, B, P, C, and D, and a segment distance d, are shown in FIG. 7A. Points B and P are the origins of the B-mode scan plane 612c and of the offset M-mode scan line 620c, respectively. Points C and D are example focal points along the offset M-mode scan line 620c. Points A and A′ are the centers of two transducers that are equidistant from point P. The distance d is the distance between any pair of adjacent transducers.

The conventional beam-forming formula for timing delays for each transducer in a flat transducer array is as follows:

Δd(x, t)=[R[t]−\sqrt{R²[t]+x²−2R[t]×Sin(θ)}]/c

where, applied to beam-forming for the offset M-mode scanning:
Δd(x, t) is the amount of delay (transmitting or receiving) for a transducer;
x is distance of the transducer along the flat array from the beam origin;
R[t] is current focal length of interest;
t is time;
θ is the angle of the scan line, namely, offset M-mode line 620c; and
c is the speed of sound in the expected target material, typically assumed to be 1540 meters/second for medical ultrasound imaging.

The delay Δd(x, t) for a transducer is essentially the expected difference in travel time to or from a focal point of interest between the transducer and the intersection of the transducer array with the scan line in question, namely, the M-mode line 620c. The derivation for the above equation can be understood with an example. Consider the point D as a focal point currently of interest at a time t (e.g., as the single transmit focal point or as the current focal point, in a series, whose echo is being received). The distance PD (from point P to point D) can be defined to be:

PD=2ndF#

where:

F# is the ratio between focal length and aperture; and

n is the number of distances d between point A (or A′) and point P.

Then, the distance AD is:

\sqrt{(2ndF#)²+(nd)²−2(2ndF#)(nd)Sin(θ)}

by trigonometry.

### Via the above equations, the desired M-mode beam may be formed.

An analysis similar to the above, using geometry, can be made for conventional beam forming for curved transducer arrays, which is schematically illustrated in FIG. 7B. FIG. 7B resembles FIG. 7A, but relates to a curved phased array. In any event, beam forming for flat phase or curved transducer arrays, in general, is known, and any such technique is adapted and employed, as mentioned above, to conduct scanning along a possibly offset M-mode line.

In addition to the M mode, Doppler-mode (D-mode) imaging is often used in conjunction with B-mode cardiology imaging in the phase array probe. D-mode medical imaging can be used, for example, in cardiology to evaluate the motion of blood through a particular part of a valve, or a particular locality within a heart. In conventional D-mode imaging, successive ultrasonic beams are sent along a single D-mode line in a B-mode plane toward the particular region of interest, referred to as the Doppler gate. Similarly to conventional M-mode imaging, the conventional D-mode line has the same position as one of the pulse lines that defines the B-mode scan plane. The echo of each D-mode beam from the Doppler-gate region is evaluated for Doppler shift by the Fast Fourier Transform (FFT) algorithm. The Doppler shift indicates the component, if any, of the velocity of the probed material in the D-mode line's direction. The velocity spectrum is plotted against time. FIG. 8 schematically illustrates an example D-mode image 210 in which the horizontal axis is the time axis. As can be seen, the shape of the example D-mode image 210 indicates a speed profile of the blood flow through the Doppler gate with the Doppler equation:

Vd=(fd*C)/2*fo*cos θ

### Where Vd: blood velocity

fd: Doppler frequency

C: Speed of sound

fo: transmit carrier

θ: incident angle

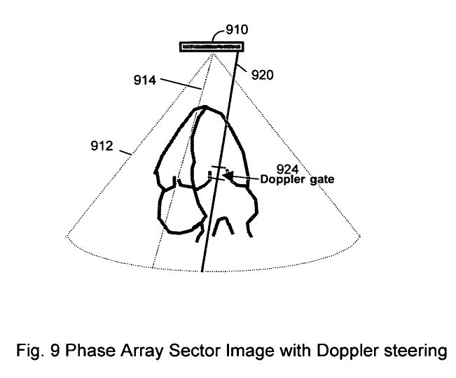

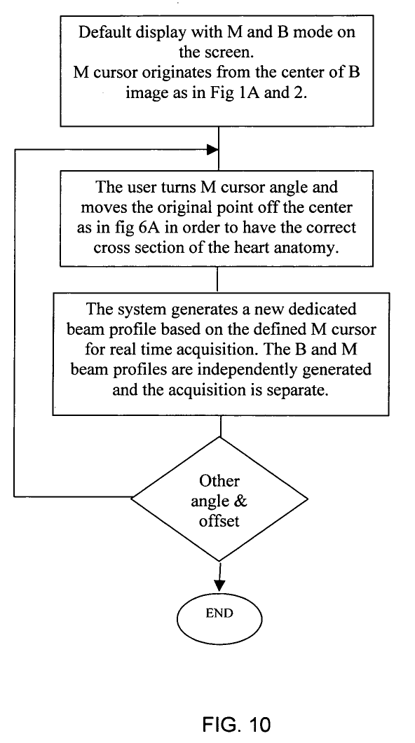

FIG. 9 is a schematic diagram of a flat array 910 of ultrasound transducer elements. The array 910 sweeps out a sector-scanning B-mode scan plane 912. The scan plane 912 includes an offset Doppler-mode line 920, according to an embodiment of the present invention. The offset Doppler-mode line 920 is used to evaluate flow velocity at a Doppler gate 924. As can be seen, the offset Doppler-mode line 920 is offset from the origin of the B-mode scan plane 912, in the sense discussed above in connection with M-mode scanning according to some embodiments of the present invention.

FIG. 10 is a schematic flow diagram that illustrates one method according to an embodiment of the present invention for conducting ultrasound imaging in a first ultrasound imaging-mode that highlights an elongated region within a larger region, the larger region being imaged according to a second ultrasound mode. For example, the first ultrasound mode may be M-mode, D-mode, or the like, and the second ultrasound mode may be B-mode, color-mode or the like.

According to above descriptions, this invention discloses a method for generating a M-mode scan line by applying an ultrasound imaging system. The method includes a step of processing a set of scanning data of the ultrasound imaging system for generating a dedicated M beam profile for constructing the M-mode line showing a time varying image with an offset of beam origin or steering angle deviated from actual scanning beams projected from the ultrasound image system whereby a requirement of first generating a B-mode profile or generating a virtual M-mode line from acquired B-mode lines are not necessary: In an exemplary embodiment, the method further includes a step of receiving a user input of the offset of beam origin or steering angle for generating the M-mode line. In an exemplary embodiment, the method further includes a step of applying the M-mode line for a cardiology diagnosis. In an exemplary embodiment, the method further includes a step of displaying the M-mode line together with a B-mode display. In an exemplary embodiment, the method further includes a step of displaying the M-mode line alone without a B-mode display. In an exemplary embodiment, the method further includes a step of moving a position and orientation of the M-Mode line in response to a biological structure. In an exemplary embodiment, the method further includes a step of associating a reference point with ultrasonic images scanned by the ultrasound image system and fixing a corresponding reference point at a chosen vertical coordinate in the M-Mode line based upon the reference point disposed at a probe aperture center. In an exemplary embodiment, the method further includes a step of implementing a flat phase array comprising a plurality of ultrasound transducer elements in the ultrasound image system. In an exemplary embodiment, the method further includes a step of receiving from a display screen as a user interface under a control of a processor of the ultrasound imaging system of a user input of the offset of beam origin or steering angle for generating the M-mode line. In an exemplary embodiment, the step of step of generating a dedicated M beam profile for constructing the M-mode line further comprising a step of using a set of delay profile with optionally gain parameters different from parameters of a B-mode beam along a nearest B-mode scan line. In an exemplary embodiment, the step of generating a dedicated M beam profile for constructing the M-mode line further comprising a step of using conducting an ultrasound scanning operation at a refresh rate substantially at or below a B-mode scanning refresh rate. In an exemplary embodiment, the step of step of generating a dedicated M beam profile for constructing the M-mode line further comprising a step of obtaining each 2D (two-dimensional) M-mode image along a time axis substantially without combining image data from ultrasonic beams separately sent along multiple scan lines intersecting M-mode scanned line at different points. In an exemplary embodiment, the step of generating a dedicated M beam profile for constructing the M-mode line further comprising a step of using a set of variable delays optionally with gains for individual transducer elements and using an offset point as the origin or center for generating the M-mode line wherein the variable delays including non-existing transducer elements disposed beyond actual edges of an phased array of the ultrasound imaging system. In an exemplary embodiment, the step of step of using the set of variable delays further includes leaving an unsymmetrical number of transducer elements to each side of a beam center of the M-mode line. In an exemplary embodiment, the method further includes a step of implementing a curved array comprising a plurality of ultrasound transducer elements in the ultrasound image system and using a phased-array beam forming algorithm to position and steer and generating the M-mode line along a user-selected input of the M-mode line. In an exemplary embodiment, the method further includes a step of generating an offset Doppler line and evaluating a flow velocity at a Doppler gate wherein offset Doppler line is offset from an origin of a B-mode scan plane. In an exemplary embodiment, the step of evaluating a flow velocity at a Doppler gate further comprising a step of evaluating a Doppler shift by a applying a Fast Fourier Transform (FFT) on echo of each D-Mode line from the Doppler gate. In an exemplary embodiment, the step of evaluating a flow velocity at a Doppler gate further comprising a step of determining a flow velocity through the Doppler gate by a Doppler equation as: Vd=(fd*C)/2*fo*cos θ; Where Vd representing the flow velocity, fd representing a Doppler frequency, C representing a speed of sound, fo representing a transmit carrier and θ resenting an incident angle.

According to above descriptions, this invention further discloses an ultrasound imaging system that includes a plurality of ultrasound transducer elements. The ultrasound imaging system further includes an M-mode processor for processing a set of scanning data of the ultrasound imaging system for generating a dedicated M beam profile for constructing the M-mode line showing a time varying image with an offset of beam origin or steering angle deviated from actual scanning beams projected from the ultrasound transducer elements whereby a requirement of first generating a B-mode profile or generating a virtual M-mode line from acquired B-mode lines are not necessary. In an exemplary embodiment, the ultrasound imaging system further includes a user interface for receiving a user input of the offset of beam origin or steering angle for generating the M-mode line.

Again, it is to be understood that the embodiments described are for the purpose of illustration and are not intended to limit the invention specifically to those embodiments.

What is claimed is:

1. A method for generating a M-mode scan line by applying an ultrasound imaging system comprising:
processing a set of scanning data of said ultrasound imaging system for generating a dedicated M beam profile for constructing said M-mode line showing a time varying image with an offset of beam origin or steering angle deviated from actual scanning beams projected from said ultrasound image system whereby a requirement of first generating a B-mode profile or generating a virtual M-mode line from acquired B-mode lines are not necessary.

2. The method of claim 1 further comprising:
receiving a user input of said offset of beam origin or steering angle for generating said M-mode line.

3. The method of claim 1 further comprising:
applying said M-mode line for a cardiology diagnosis.

4. The method of claim 1 further comprising:
displaying said M-mode line together with a B-mode display.

5. The method of claim 1 further comprising:
displaying said M-mode line alone without a B-mode display.

6. The method of claim 1 further comprising:
moving a position and orientation of said M-Mode line in response to a biological structure.

7. The method of claim 1 further comprising:
associating a reference point with ultrasonic images scanned by said ultrasound image system and fixing a corresponding reference point at a chosen vertical coordinate in the M-Mode line based upon said reference point disposed at a probe aperture center.

8. The method of claim 1 further comprising:
implementing a flat phase array comprising a plurality of ultrasound transducer elements in said ultrasound image system.

9. The method of claim 1 further comprising:
receiving from a display screen as a user interface under a control of a processor of said ultrasound imaging system of a user input of said offset of beam origin or steering angle for generating said M-mode line.

10. The method of claim 1 wherein:
said step of generating a dedicated M beam profile for constructing said M-mode line further comprising a step of using a set of delay profile with optionally gain parameters different from parameters of a B-mode beam along a nearest B-mode scan line.

11. The method of claim 1 wherein:
said step of generating a dedicated M beam profile for constructing said M-mode line further comprising a step of using conducting an ultrasound scanning operation at a refresh rate substantially at or below a B-mode scanning refresh rate.

12. The method of claim 1 wherein:
said step of generating a dedicated M beam profile for constructing said M-mode line further comprising a step of obtaining each 2D (two-dimensional) M-mode image along a time axis substantially without combining image data from ultrasonic beams separately sent along multiple scan lines intersecting M-mode scanned line at different points.

13. The method of claim 1 wherein:
said step of generating a dedicated M beam profile for constructing said M-mode line further comprising a step of using a set of variable delays optionally with gains for individual transducer elements and using an offset point as the origin or center for generating the M-mode line wherein said variable delays including non-existing transducer elements disposed beyond actual edges of an phased array of said ultrasound imaging system.

14. The method of claim 13 further comprising:
said step of using said set of variable delays further includes leaving an unsymmetrical number of transducer elements to each side of a beam center of said M-mode line.

15. The method of claim 1 further comprising:
implementing a curved array comprising a plurality of ultrasound transducer elements in said ultrasound image system and using a phased-array beam forming algorithm to position and steer and generating said M-mode line along a user-selected input of said M-mode line.

16. The method of claim 1 further comprising:
generating an offset Doppler line and evaluating a flow velocity at a Doppler gate wherein offset Doppler line is offset from an origin of a B-mode scan plane.

17. The method of claim 16 wherein:
said step of evaluating a flow velocity at a Doppler gate further comprising a step of evaluating a Doppler shift by a applying a Fast Fourier Transform (FFT) on echo of each D-Mode line from said Doppler gate.

18. The method of claim 1 wherein:
said step of evaluating a flow velocity at a Doppler gate further comprising a step of determining a flow velocity through said Doppler gate by a Doppler equation as:
Vd=(fd*C)/2*fo*cos θ
Where Vd representing said flow velocity, fd representing a Doppler frequency, C representing a speed of sound, fo representing a transmit carrier and θ resenting an incident angle.

19. An ultrasound imaging system comprising a plurality of ultrasound transducer elements and said ultrasound imaging system further comprising:
an M-mode processor for processing a set of scanning data of said ultrasound imaging system for generating a dedicated M beam profile for constructing said M-mode line showing a time varying image with an offset of beam origin or steering angle deviated from actual scanning beams projected from said ultrasound transducer elements whereby a requirement of first generating a B-mode profile or generating a virtual M-mode line from acquired B-mode lines are not necessary.

20. The ultrasound imaging system of claim 19 further comprising:
a user interface for receiving a user input of said offset of beam origin or steering angle for generating said M-mode line.
